(19) United States
(12) Patent Application Publication
(10) Pub. No.: US 2022/0354655 A1
(43) Pub. Date: Nov. 10, 2022

(54) FEMORAL NECK-PRESERVING ARTIFICIAL HIP JOINT FEMORAL PROSTHESIS

(71) Applicant: Xieping DONG, Nanchang (CN)

(72) Inventors: Xieping DONG, Nanchang (CN); Qingli LI, Nanchang (CN); Zizheng AI, Nanchang (CN); Jingna LI, Yongzhou (CN); Weiyi HE, Guangzhou (CN)

(21) Appl. No.: 17/631,056

(22) PCT Filed: Oct. 12, 2020
(86) PCT No.: PCT/CN2020/120505

§ 371 Date: Jan. 28, 2022

(51) Int. Cl. A61F 2/36

(52) U.S. Cl. CPC A61F 2/3601; A61F 2002/3611; A61F 2002/30934

(57) ABSTRACT

The present disclosure comprises a ball head, a neck part, a cover body, and a fastener, wherein the cover body is in a shape of a thin-walled cup, including a cup buckled and embedded on the femoral neck left after the femoral head is removed and a circle of skirt plates attached to the lower edge of the cup to extend the covering range of the cup to the femoral neck and the intertrochanteric femur; a through hole for a cable to penetrating through is formed in the tail end of each skirt plate, and a limiting clip for limiting the cable for cerclage of the cup is arranged outside the skirt plate; and then stable rigid connection is conducted using a compression ring; and the fastener comprises a screw or an additional perforated steel plate and a cable which is fixed in a cerclage and tension manner.

### TECHNICAL FIELD

The present disclosure relates to the field of medical devices, and in particular relates to a femoral neck-preserving artificial hip joint femoral prosthesis.

### BACKGROUND ART

The artificial hip joint is divided into a total hip joint and a hemi-hip joint. The hemi-hip joint, also known as the artificial femoral head, is used to replace the damaged femoral head in the human body; the total hip joint is divided into two major parts: the femoral prosthesis and the acetabular prosthesis. At present, the extensively applied artificial femoral head or artificial hip joint femoral prosthesis is basically made of a ball head part, a neck part, and a stem part, wherein the size of the ball head of the hemi-hip joint must be in fit with undamaged acetabulum of the human body, and the ball head part of the total hip joint must be in fit with a liner of the acetabular prosthesis. Leaving the acetabular prosthesis aside, both the femoral prosthesis of the total hip joint and the hemi-hip joint must be stably combined by amputating the femoral neck, hollowing out cancellous bone/marrow cavity at trochanter region of femur and then inserting the stem part into the cancellous bone/marrow cavity. However, there area two defects in such structure and combination mode, one is that it is not the optimal mode in biomechanics, the gravity of the human body is basically and directly transferred from the femoral stem to the middle-upper femur shaft which is most tightly combined with the femoral stem, stress stimulation on the trochanter region of the femur surrounding the upper end of the femoral stem inevitably leads to disuse osteoporosis, thus resulting in prosthesis loosening; secondly, the cancellous bone at the osteotomy section of the femoral neck is inevitably exposed, and when a human defense system such as giant cells engulfs and removes fine particles generated by mutual abrasion of the artificial femoral head and the acetabulum, the exposed cancellous bone is engulfed together, resulting in osteolysis, and also leading to prosthesis loosening. Symptoms such as pain and dysfunctions caused by prosthesis loosening may force the patient to undergo another joint replacement (revision) surgery.

Stem-free femoral prostheses are disclosed by CN2318995, CN2430959, CN2566818, CN100374093 in succession, such stem-free joints can and must preserve the femoral neck, which can overcome the defects of the above-mentioned artificial joins with stems. The stem-free joint has the common characteristics that the stem-free joint is of a circular bell-shaped collar structure, is attached to the femoral neck by being embedded with the cylindrical femoral neck sawed by a trephine, and is reinforced on the femoral neck by the central screw screwed in along the axis or by adding peripheral screws, a reinforcing plate and the like. However, due to the fact that the anatomical form of the femoral neck is not regular, in order to obtain the cylindrical femoral neck which can be tightly embedded with the inner cavity of the prosthesis and is long enough, the most solid femoral calcar (located at the lower part of the femoral neck) in the femoral neck is sawed off in addition to sawing off bone cortex at the upper edge and the lower edge of the femoral neck, resulting in great weakening of the strength of the base to which the prosthesis is attached. Therefore, the stability of the prosthesis is seriously weakened, and the stability is more affected when the proximal femur is more osteoporotic. Hence, in such type of stem-free joint, there have been a situation that the stem-free joint has to be replaced with the hip joint with a stem due to the fact that the femoral neck is hammered off in the process of installing the stem-free joint during operation, and a situation of early postoperative loosening caused by insufficient basic strength.

### SUMMARY

The present disclosure is to provide a femoral neck-preserving artificial hip joint femoral prosthesis capable of obtaining strong enough initial stability without damaging femoral calcar. The technical solution adopted by the present disclosure is as follows: a femoral neck-preserving artificial hip joint femoral prosthesis comprises a ball head, a neck part, a cover body, and a fastener, wherein the cover body is in a shape of a thin-walled cup, including a cup buckled and embedded on the femoral neck left after the femoral head is removed and a circle of skirt plates attached to the lower edge of the cup to extend the covering range of the cup to the femoral neck and the intertrochanteric of femur; the cup and the skirt plates are of an integrated structure or of a detachable split structure; the fastener comprises a screw or a cable for reinforcing the connection of the cover body and the femoral neck.

The ball head is a spherical body in fit with a human acetabulum or an artificial acetabulum liner, and an inward concave conical blind cavity or a downward-convex neck part is axially formed in a base of the ball head; the cup is provided with an upward-convex neck part inserted into the inward-concave blind cavity of the ball head, or is provided with an inward-concave conical bearing cavity for bearing the downward-convex neck of the ball head; the upward-convex neck part or the inward concave conical bearing cavity of the cup is a hollow conical body with two through ends; the skirt plates are attached to the lower edge of the cup in a spaced manner, are of a sheet structure, and can be bent during an operation to adapt to the shape of a bone surface.

A through hole which penetrates through the skirt plate for a cable to penetrate through is formed in the tail end of the skirt plate, and a limit clip for limiting a position of the cable for the cerclage of the cover body can be additionally arranged on the outer side surface of the skirt plate.

When the cup and the skirt plate are of a detachable split structure, the skirt plate with proper length can be selected according to intraoperative conditions to be inserted into the peripheral wall of the cup in the corresponding direction to form stable rigid connection.

Through grooves are formed in the outer edge of the top end of the cup in a spaced manner, and a notch is formed in the lower edge of the cup below each through groove; the top end of the skirt plate is folded to be in a hook shape, an inward protruding point is arranged below the hook, and when the hook is inserted into the through groove, the inward protruding point is just clamped into the notch.

A compression ring capable of being closely connected to the top end of the cup can be additionally arranged at the top end of the cup, the bottom surface of the compression ring just presses the top end of the skirt plate, and the fastened compression ring can enhance the connection stability of the cup and the skirt cup.

The screw comprises a central screw penetrating through a middle axle of the cup and auxiliary screws at two sides, and the central screw and the auxiliary screws are arranged in parallel; the central screw has two structural forms, i.e., an antegrade central screw screwed into the bone from the cup and a reverse central screw screwed into the cup from the lateral femur and screwed with the cup.

Two auxiliary screw holes are formed in the edges of the two sides of the axis of the central screw at the top end of the cup, and the screw heads of the two auxiliary screws screwed into the bone through the two auxiliary screw holes can press the compression ring to prevent the compression ring from loosening.

The nuts and washers or a perforated steel plate in fit with the screws can be additionally arranged at the positions, penetrating through the lateral femur, of the central screw and the auxiliary screws, the perforated steel plate is provided with through holes for the central screw and the auxiliary screws to penetrate through, the through hole is in a long groove shape with the two arc ends, and the lower end of the steel plate can also be provided with screw holes used for fixing the steel plate to the upper end of the femoral shaft.

The connection form of the cable and the cup has two forms, i.e., a cerclage mode of annularly binding after encircling around the limiting clips of all skirt plates, and a tension mode of tying the cable penetrating through the through holes in the tail ends of the skirt plates to the screws penetrating through the lateral femur through the bone tunnels.

The present disclosure has the beneficial effects that the femoral neck-preserving artificial hip joint femoral prosthesis capable of obtaining strong enough initial stability without damaging the femoral calcar can be attached to the proximal femur in a femoral neck-preserving fixing mode. The femoral neck-preserving artificial hip joint femoral prosthesis is simple and reasonable in structure, and has five-micro features, i.e., micro surgical injury, micro stress shielding, micro osteolysis, minimally invasive revision, and micro age limit, wherein the micro surgical injury is shown as small incision replacement, small tissue injury, quick postoperative recovery, preservation of femoral neck, preservation of trochanter region and cancellous bone in the medullary cavity of the proximal femur, creation of excellent conditions for later femoral revision, no medullary cavity filing, no stem inserting fixation, and reduction of complications such as postoperative medullary cavity bleeding, fat embolism, bone cement reaction, medullary cavity infection, loosening, sinking, fracture of the artificial stem, fracture of femoral shaft and the like; the micro stress shielding is shown as high stress concentration on a prosthesis stem of the conventional joint with a stem, tiny stress and high stress shielding on the proximal femur cortex, tiny stress of the cup, the central screw, and the cancellous bone screw, high stress concentration on the femoral neck cortex, and relatively high stress concentration and slight stress shielding at the proximal femur, wherein the gravity of the human body can be completely transferred to femoral neck bone cortex below the cup through the cup and is naturally transited to the bone cortex of the proximal femur, and stress shielding and secondary periprosthesis osteoporosis phenomena hardly occur; the micro osteolysis shows that a small amount of cancellous bone exposed after femoral neck osteotomy is basically closed after being sleeved with the cup, polyethylene abrasive particles are difficult to be in contact with the cancellous bone, and osteolysis caused in the particle phagocytosis process can be effectively controlled by tissues; the minimally invasive revision shows that the proximal femur can be kept at a relative normal bone density after being replaced with the stem-free joint, the dissolved bone is less, the service life of the joint can be prolonged, the stem-free joint can be continuously used for revision even if revision is needed due to loosening and more residual bone structures exist, and small-incision minimally invasive revision can still be used; and the micro age limit shows that the joint is suitable for low-age adult patients, and due to micro stress shielding and low osteolysis, the service life of the joint is longer than that of the joint with the stem which is made of the same material and is the same as the acetabulum prosthesis after primary replacement; and the difficulty of minimally invasive revision is reduced.

### BRIEF DESCRIPTION OF THE DRAWINGS

FIG. 1 is a two-dimensional diagram of an embodiment I of the present disclosure;

FIG. 2 is a diagram of an outline effect of an embodiment I of the present disclosure;

FIG. 3 is a diagram of a breakdown effect of an embodiment I of the present disclosure;

FIG. 4 is a two-dimensional diagram of an embodiment II of the present disclosure;

FIG. 5 is a diagram of an outline effect of an embodiment II of the present disclosure;

FIG. 6 is a diagram of a breakdown effect of an embodiment II of the present disclosure;

FIG. 7 is a two-dimensional diagram of an embodiment III of the present disclosure;

FIG. 8 is a diagram of clinical application of an embodiment II of the present disclosure;

The structures and serial numbers of the present disclosure in the drawings:

1-ball head, 11-inward-concave conical blind cavity 11, downward-convex neck part 12;

2- neck part;

3-cover body, 31-cup, 311-top end of cup, 312-lower edge of cup, 313-through groove, 314-gap, 315-notch, 316-upward-convex neck part, 317-inward-concave conical bearing cavity, 318-auxiliary screw hole, 32-skirt plate, 321-through hole, 322-limiting clip, 323-protruding point, 324-hook, 33-compression ring;

4-screw, 41-central screw, 411-anterograde central screw 411, 412-reverse central screw, 42-nut, 43-auxiliary screw, 44-perforated steel plate, 441-screw through hole 441, 441-femoral shaft screw through hole, 45-femoral shaft screw 45;

5-cable, 51-cerclage cable, 52-tension cable.

### DETAILED DESCRIPTION OF THE EMBODIMENTS

The present disclosure can be achieved through the following embodiments.

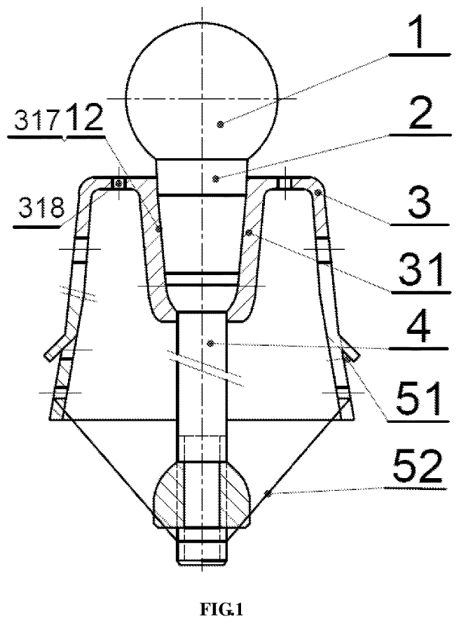

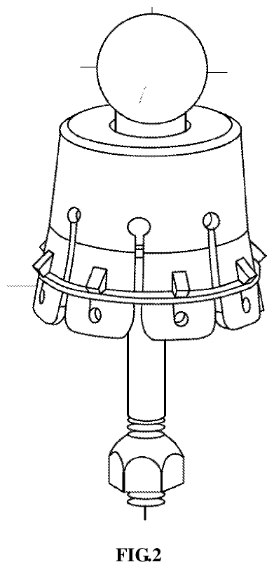

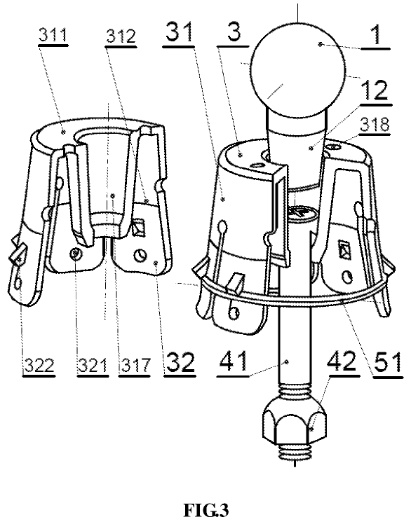

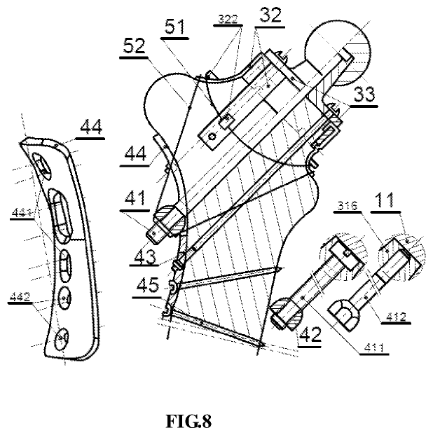

Embodiment I: as shown in FIG. 1, FIG. 2, FIG. 3, and FIG. 8, an integrated femoral neck-preserving artificial hip joint femoral prosthesis comprises a ball head 1, a neck part 2, a cover body 3, and a fastener.

The ball head 1 is a spherical body in fit with a human acetabulum or an artificial acetabulum liner, and an inward-concave conical blind cavity 11 or a downward-convex neck part 12 is axially formed in a base of the ball head 1.

The ball head 1 is a spherical body in fit with a human acetabulum or an artificial acetabulum liner, and an inward-concave conical blind cavity 11 or a downward-convex neck part 12 is axially arranged at a base of the ball head 1.

The cover body 3 is in a shape of thin-walled cup, including a rigid cup 31 and 4-10 pieces of skirt plates 32 of a rigid yieldable sheet structure; the skirt plates 32 are of a sheet-shaped structure and can be bent during an operation to adapt to the shape of the bone surface; the number of the skirt plates 32 is preferably 8, and the skirt plates 32 are dispersed and attached to the lower edge 312 of the cup in an axial direction; and the cup 31 and the skirt plates 32 are of an integral structure, which are buckled and embedded on the femoral neck left after the femoral head is removed.

Further, the inner surfaces of the cup 31 and the skirt plate 32 are both rough surfaces beneficial to bone growth and even porous surfaces beneficial to bone ingrowth.

Further, the cup 31 is provided with an upward-convex neck part 316 inserted into the inward-concave conical blind cavity 11 of the ball head, or is provided with an inward-concave conical bearing cavity 317 capable of bearing a downward-convex neck part 12 of the ball head 1; and the upward-convex neck part 316 or the inward concave conical bearing cavity 317 of the cup 31 is a hollow cone with two through ends.

The neck part 2 is a part integrated into the ball head 1 or the cup 31, i.e., which is the downward-convex neck part 12 of the ball head 1, or the upward-convex neck part 316 of the cup 31.

A through hole 321 for a cable 5 to penetrate through is formed in the tail end of the skirt plate 32, a limiting clip 322 for limiting the position of the cable 5 for cerclage of the cover body 3 can be additionally arranged at the outer surface of the skirt plate 32; the skirt plates 32 are arranged from the upper edge of the femoral neck to the lower edge of the femoral neck, with the lengths sequentially changed to encompass the base of the femoral neck as suitable.

The fastener comprises a screw 4 and a cable 5 for reinforcing the connection of the cover body 3 and the femoral neck.

The screw 4 comprises a central screw 41 penetrating through the middle axle of the cover body 3 and fastening the cover body to the femoral neck; the central screw 41 comprises a screw head and a screw rod, the central screw 41 is fixed in two forms: an anterograde central screw 411 penetrating into the cup 31 obliquely from the inner top to the outer bottom to be fastened to the bone of the proximal femur, and a reverse central screw 412 penetrating into bone obliquely from outer bottom of the trochanter major of femur to the inner top to be fastened to the cup 31.

When the anterograde central screw 411 is adopted, the outer surface of the screw head is smooth and free of threads, and the end part of the screw head is axially provided with a screwdriver connector in hexagonal or star, torx, slotted, cross and other shapes. The screw rod may be fully threaded or only end threaded, and the thread form may be a bone thread or a standard thread; the anterograde central screw 411, after penetrating through a hollow cavity of an upward-convex neck part 316 or an inward-concave conical bearing cavity 317 of the cup 31, penetrates into the middle axle of the femoral neck from outer top to inner bottom till to penetrate through the bone cortex at the outer bottom of the trochanter major of the femur. To enhance fixation effect to the patient with osteoporosis of proximal femur, the fastening can be achieved by screwing a nut 42 and the antegrade central screw 411 having the standard threaded screw rod in opposite.

When the reverse central screw 412 is adopted, the tail end of the screw rod of the reverse central screw 412 is provided with standard threads, and is screwed with the hollow cavity of the cup 31.

Further, the screw 4 further comprises two auxiliary screws 43 located at two sides of the central screw 41, and the two auxiliary screws 43 are preferably locking screws; correspondingly, two auxiliary screw holes 318 which are located at the two sides of the axis of the central screw 41 and arranged parallel to the axis of the central screw 41 are formed in the edges, with respect to the upper edge and the lower edge of the femoral neck, of the top end of the cup 31; preferably, the two auxiliary screw holes 318 are symmetrically arranged at two sides of the axis of the central screw 41. During operation, the two auxiliary screw holes 318 are preferably formed in the upper edge and the lower edge of the femoral neck respectively, and after the auxiliary screws 43 are screwed into the two auxiliary screw holes 318, the tensile strength of the upper edge of the femoral neck and the compressive strength of the lower edge of the femoral neck (femoral calcar) can be increased, and an anti-rotation effect is enhanced.

When the cover body is fastened in a mode of screwing the nut 42 and the anterograde central screw 411 having a standard threaded screw rod in opposite, in order to enhance the fixing stability, especially the initial stability, for the patient with osteoporosis of proximal femur, washers of screws 4 or a perforated steel plate 44 allowing the central screw 41 and the auxiliary screw 43 to penetrate through jointly can be additionally arranged below the trochanter major of the lateral femur to disperse the stress borne by screw roads, especially outlets, of the central screw 41 and the auxiliary screws 43 on the lateral femur, thus preventing loss of fixation strength due to compression of the bone.

Due to the fact that an included angle between the femoral shaft axis and the femoral neck axis, i.e., a collodiaphyseal angle, is between 127-135 degrees, the hole spacing of the perforated steel plate 44 clinging to the outer edge of the femur to allow the central screw 41 and the auxiliary screw 43 in parallel arrangement to jointly penetrate through is necessarily larger than the hole spacing of the screws at the top end 311 of the cup. To ensure that the perforated steel plate 44 can adapt to people with different body types, screw through holes 441, for bearing the central screw 41 and the auxiliary screw 43, on the perforated steel plate 44 are preferably arranged to be in the shape of a long groove with two arc-shaped ends.

Further, the screw 4 further comprises a femoral shaft screw 45, 1-3 femoral shaft screw through holes 442 can be provided on the lower end of the perforated steel plate 44, preferably locking screw holes, and after the femoral shaft screws 45 are screwed into the femoral shaft screw through holes 442, the fixation effect can be strengthened by means of the cortical bone at the upper end of the femoral shaft having a bone mineral density and strength much higher than those of the cancellous bone.

The fastening to the cover body 3 by the cable 5 comprises two modes: a cerclage mode, and a tension mode.

The cerclage cable 51 is sequentially limited on the limiting clips 322 of all the skirt plates 32 for ring binding to make the skirt plates 32 be bent to cling to the femoral neck, thus acquiring firm initial stability, and facilitating the cancellous bone in the femoral neck subjected to decortication treatment during operation to grow onto or grow into the surface of the cover body 3 to form permanent biological fixation.

Bony tunnels are drilled at the upper edge of an outlet below the trochanter major of the femur from the through holes 321 in the tail ends of two skirt plates 32 located on the front top and the rear top of the femoral neck towards the central screw 41, and then bony tunnels are drilled at the lower edge of the outlet below the trochanter major of the femur from the through holes 321 in the tail ends of two skirt plates 32 located on the front bottom and the rear bottom of the femoral neck towards the central screw 41; two ends of one tension cable 5 are used to respectively drill into corresponding bony tunnels below the two skirt plates 32 from through holes 321 in the tail ends of the two skirt plates 32 at the front top and the rear top of the femoral neck, and get out of a bony tunnel outlet of the central screw 41 at the upper edge of the outlet below the trochanter major of the femur, then bypass the tail end of the central screw 41 from the front side and the rear side respectively to drill into two bony tunnels of the central screw 41 at the lower edge of the outlet below the trochanter major of the femur, and then get out of the through holes 321 in the tail ends of two skirt plates 32 at the front bottom and the rear bottom of the femoral neck, and after the two ends of the tension cable 52 are tightened and are connected into a whole by using a fixing clamp attached to the tension cable 52, the stability of the cover body 3 can be enhanced by tension.

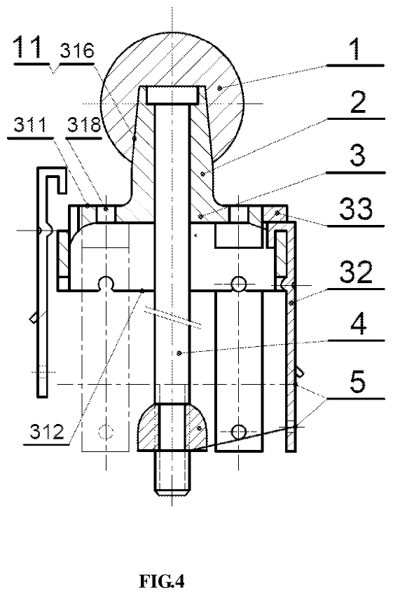

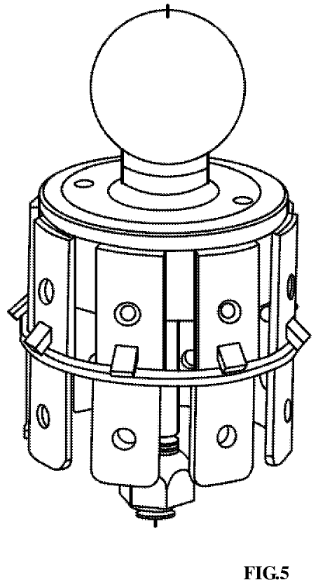

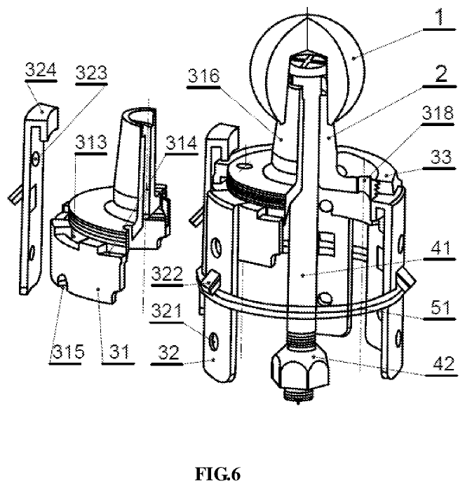

Embodiment II: as shown in FIG. 4, FIG. 5, FIG. 6, and FIG. 8, a split type femoral neck-preserving artificial hip joint femoral prosthesis comprises a ball head 1, a neck part 2, a cup 31, a skirt plate 32, a compression ring 33, and a fastener.

The ball head 1, the neck part 2 and the fastener of the split type femoral neck-preserving artificial hip joint femoral prosthesis in the embodiment II are same as those of the integrated femoral neck-preserving artificial hip joint femoral prosthesis in the embodiment I, the difference is that the cover body 3 is of a split structure, the cup 31 and the skirt plate 32 are of a detachable split structure, the skirt plate 32 with proper length can be selected according to intraoperative conditions to be inserted into the peripheral wall of the cup 31 in the corresponding direction; and the split type cup 31 is also axially provided with a hollow upward-convex neck part 316 or an inward-concave conical bearing cavity 317.

Through grooves 313 with the same width as the skirt plates 32 are arranged at the outer edge of the top end 311 of the cup in a spaced manner, the lower edge 312 of the cup below each through groove 3131 is provided with a notch 315, the top end of the skirt plate 32 is folded to be in a shape of hook 324, and an inward protruding point 323 is arranged below the hook 324; when the hook 324 is inserted into the through groove 313, the inward protruding point 323 is just clamped into the notch 315, and the skirt plate 32 forms a stable rigid connection with the cup 31 at the moment.

To enhance the stability of the cup 31 and the skirt plate 32, a compression ring 33 capable of being closely connected to the top end 311 of the cup can be additionally arranged at the top end 311 of the cup; at the moment, an axial section of the cup 31 can be changed into a convex shape of the split type cup 31 from a square shape of an integrated cup 31, thus making the split type cup 31 become a coaxial cylindrical shape with a small top and large bottom; the outer edge of the small cylinder is provided with threads, and 4-10, preferably 8, gaps 314 with the same width as the skirt plate 32 are formed in the top end of the large cylinder in a spaced manner and clung to the outer edge of the small cylinder; and the inner edge of the gap 314 removes exactly the top end of the cup 31 at the same time and forms an axial through groove 313, preferably, the gaps 314 are equally spaced.

Further, the compression ring 33 is an annular nut with the outer diameter equal to that of a large cylinder of the cup 31, the height equal to that of a small cylinder of the cup 31, and an inner ring in thread fit with the outer edge of the small cylinder; and after being screwed with the small cylinder, the bottom surface of the compression ring 33 just presses the top end of the skirt plate 32, and the cup 31 and the skirt plate 32 are closely connected after the compression ring 33 is fastened under the limitation of the gap 314 and the notch 315 at the lower edge of the cup 31.

The skirt plate 32 under the protruding point 322 and the skirt plate 32 of the integrated cover body 3 have the same shape structures but different lengths, the length can be flexibly selected according to the body type of the patient and the specific orientation used during the operation to achieve the optimal covering and fixing effects.

Further, the fastener further comprises two auxiliary screws 43, preferably locking screws; correspondingly, the top end 311 of the cup is equivalent to two auxiliary screw holes 318, formed at the two sides of the middle axle of the cup 31 and parallel to the middle axle, at the top end of the inner edge of the small cylinder; the two auxiliary screw holes 318 are respectively formed at the upper edge and the lower edge of the femoral neck during operation, and after the auxiliary screws 43 are screwed into the two auxiliary screw holes 318, the tensile strength of the upper edge of the femoral neck and the compressive strength of the lower edge (femoral calcar) of the femoral neck can be increased, the anti-rotation effect is enhanced; and screw heads of the two auxiliary screws 43 can press the compression ring 33 tightly to prevent the phenomenon that the fixing effect of the skirt plates 32 on the femoral neck is weakened due to loosening and retreating of the compression ring 33.

Other fasteners and structural features thereof are same as those of the integrated femoral neck-preserving artificial hip joint femoral prosthesis in the embodiment I.

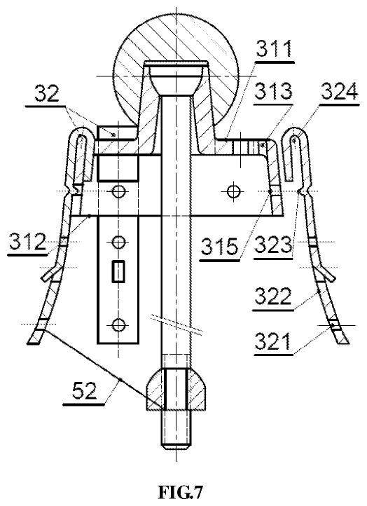

Embodiment III: as shown in FIG. 7 and FIG. 8, a split type femoral neck-preserving artificial hip joint femoral prosthesis comprises a ball head 1, a neck part 2, a cup 31, a skirt plate 32, and a fastener.

The split type femoral neck-preserving artificial hip joint femoral prosthesis in the embodiment III is basically same as the split type femoral neck-preserving artificial hip joint femoral prosthesis in the embodiment II, and the cover body 3 is also in a split type; the two embodiments are characterized in that the cup of the embodiment III is not provided with the compression ring 33 of the embodiment II, thus the axial section of the cup 31 is in the square shape of an integrated cup 31, and then the through grooves 313 formed in the outer edge of the top end 311 of the cup in a spaced manner are not provided with gaps 314.

In the embodiment III, relatively firm initial stability can also be acquired under the fastening of the screw and the cable, however, the fixation strength is slightly inferior to that in the embodiment II.

The application of the present disclosure has clearly and completely described the innovative ideas of the present disclosure, however, apparently, the described embodiments are only a part rather than all of the embodiments of the present disclosure. Based on the embodiments in the present disclosure, all changes, modifications, replacements, variations, and other embodiments obtained by those of ordinary skill in the art without creative efforts shall fall within the scope of protection of the present disclosure.

1. A femoral neck-preserving artificial hip joint femoral prosthesis, comprising:
a ball head;
a neck part;
a cover body; and
a fastener;
wherein the cover body is in a shape of a thin-walled cup, including a cup buckled and embedded on the femoral neck left after the femoral head is removed and a circle of skirt plates attached to the lower edge of the cup to extend the covering range of the cup to the femoral neck and the intertrochanteric femur; the cup and the skirt plates are of an integrated structure or a detachable split structure;
wherein the fastener comprises a screw or a cable for reinforcing the connection of the cover body and the femoral neck.

2. The femoral neck-preserving artificial hip joint femoral prosthesis according to claim 1, wherein the ball head is a spherical body in fit with a human acetabulum or an artificial acetabulum liner, and an inward-concave conical blind cavity or a downward-convex neck part is axially formed on a base of the ball head; the cup is provided with an upward-convex neck part inserted into the inward-concave blind cavity of the ball head, or is provided with an inward-concave conical bearing cavity for bearing the downward-convex neck of the ball head; the upward-convex neck part or the inward-concave conical bearing cavity of the cup is a hollow cone with two through ends; the skirt plates are attached to the lower edge of the cup in a spaced manner, are of a sheet structure, and can be bent during operation to adapt to the shape of a bone surface.

3. The femoral neck-preserving artificial hip joint femoral prosthesis according to claim 2, wherein a through hole which penetrates through the skirt plate for the cable to penetrated through is formed in the tail end of the skirt plate, and a limit clip for limiting a position of the cable for the cerclage of the cover body can be additionally arranged on the outer side surface of the skirt plate.

4. The femoral neck-preserving artificial hip joint femoral prosthesis according to claim 3, wherein when the cup and the skirt plate are of a detachable split structure, the skirt plate with proper length can be selected according to intraoperative conditions to be inserted into the peripheral wall of the cup in the corresponding direction to form stable rigid connection.

5. The femoral neck-preserving artificial hip joint femoral prosthesis according to claim 4, wherein through grooves are formed in the outer edge of the top end of the cup in a spaced manner, and a notch is formed in the lower edge of the cup below each through groove; the top end of the skirt plate is folded to be in a hook shape, an inward protruding point is arranged below the hook, and when the hook is inserted into the through groove, the inward protruding point is just clamped into the notch.

6. The femoral neck-preserving artificial hip joint femoral prosthesis according to claim 5, wherein a compression ring capable of being closely connected to the top end of the cup can be additionally arranged at the top end of the cup, the bottom surface of the compression ring just presses the top end of the skirt plate, and the fastened compression ring can enhance the connection stability of the cup and the skirt plate.

7. The femoral neck-preserving artificial hip joint femoral prosthesis according to claim 1, wherein the screw comprises a central screw penetrating through a middle axle of the cup and auxiliary screws at two sides, and the central screw and the auxiliary screws are arranged in parallel; the central screw has two structural forms, including an antegrade central screw screwed into the bone from the cup and a reverse central screw screwed into the cup from the lateral femur and screwed with the cup.

8. The femoral neck-preserving artificial hip joint femoral prosthesis according to claim 1, wherein two auxiliary screw holes are formed in the edges of the two sides of the axis of the central screw at the top end of the cup, and the screw heads of the two auxiliary screws screwed into the bone through the two auxiliary screw holes can press the compression ring to prevent the compression ring from loosening.

9. The femoral neck-preserving artificial hip joint femoral prosthesis according to claim 1, wherein the nuts and washers or a perforated steel plate in fit with the screws can be additionally arranged at the positions, penetrating through the lateral femur, of the central screw and the auxiliary screws, the perforated steel plate is provided with through holes for the central screw and the auxiliary screws to penetrate through, the through hole is in a long groove shape with the two arc ends, and the lower end of the steel plate can also be provided with screw holes used for fixing the steel plate to the upper end of the femoral shaft.

10. The femoral neck-preserving artificial hip joint femoral prosthesis according to claim 1, wherein the connection form of the cable and the cup has two forms, i.e., a cerclage mode of annularly binding after encircling around the limiting clips of all skirt plates, and a tension mode of tying the cable penetrating through the through holes in the tail ends of the skirt plates to the screws penetrating through the lateral femur through the bone tunnels.

11. The femoral neck-preserving artificial hip joint femoral prosthesis according to claim 6, wherein two auxiliary screw holes are formed in the edges of the two sides of the axis of the central screw at the top end of the cup, and the screw heads of the two auxiliary screws screwed into the bone through the two auxiliary screw holes can press the compression ring to prevent the compression ring from loosening.

12. The femoral neck-preserving artificial hip joint femoral prosthesis according to claim 7, wherein two auxiliary screw holes are formed in the edges of the two sides of the axis of the central screw at the top end of the cup, and the screw heads of the two auxiliary screws screwed into the bone through the two auxiliary screw holes can press the compression ring to prevent the compression ring from loosening.

13. The femoral neck-preserving artificial hip joint femoral prosthesis according to claim 7, wherein the nuts and washers or a perforated steel plate in fit with the screws can be additionally arranged at the positions, penetrating through the lateral femur, of the central screw and the auxiliary screws, the perforated steel plate is provided with through holes for the central screw and the auxiliary screws to penetrate through, the through hole is in a long groove shape with the two arc ends, and the lower end of the steel plate can also be provided with screw holes used for fixing the steel plate to the upper end of the femoral shaft.
